(19) United States
(12) Patent Application Publication
(10) Pub. No.: US 2019/0328846 A1
(43) Pub. Date: Oct. 31, 2019

(54) PHARMACEUTICAL COMPOSITIONS FOR THE TREATMENT OF RETINAL DEGENERATIVE DISEASES

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); SORBONNE UNIVERSITE, PARIS (FR)

(72) Inventors: Thierry LEVEILLARD, Paris (FR); Géraldine MILLET-PUEL, Paris (FR); Alexandra Lyor BOUAZIZ, (US)

(21) Appl. No.: 16/463,245

(22) PCT Filed: Nov. 30, 2017
(86) PCT No.: PCT/EP2017/080956

§ 371 Date: May 22, 2019

(30) Foreign Application Priority Data
Dec. 1, 2016 (EP) 16306592.3

(51) Int. Cl. A61K 38/45; C12N 9/12; A61K 9/00; A61P 27/02; C12N 15/86

(52) U.S. Cl. CPC A61K 38/45; C12N 9/1205; A61K 9/0048; C12Y 207/01105; A61P 27/02; C12N 15/86; A61K 9/0019

(57) ABSTRACT

The present invention relates to methods and pharmaceutical compositions for the treatment of retinal degenerative diseases. The inventors identified a new key actor of the mechanism underlying the protective role of RdCVF: 6-phosphofructo-2-kinase/fructose-2,6-bisphosphatase 2 (PFKFB2). The inventors showed that PFKFB2 is expressed by cones in a rod-dependant manner. In particular, they showed that its expression follows the viability of cones: its expression is lost in an animal model retinitis pigmentosa. The inventors accumulated evidences that PFKFB2, especially its kinase domain, is involved in the mechanism of action of RdCVF. More particularly they showed that transduction of a polynucleotide encoding for PFKFB2 increases cone survival. In particular, the present invention relates to a method of treating a retinal degenerative disease in a subject in need thereof comprising administering to the subject a therapeutically effective amount of a polynucleotide encoding for the PFKFB2 kinase domain.

### FIELD OF THE INVENTION

The present invention relates to methods and pharmaceutical compositions for the treatment of retinal degenerative diseases.

### BACKGROUND OF THE INVENTION

RdCVF, a truncated thioredoxin-like protein lacking thioloxidoreductase activity, was identified by high content screening of a mouse retinal cDNA library on cone-enriched cultures from chicken embryos (Leveillard et al., 2004). RdCVF is an alternative splice variant of the nucleoredoxin-like 1 (Nxn11) gene, whose other splice product is RdCVFL, an active thioredoxin that protects its binding partner, the microtubule associated protein TAU, from oxidation and aggregation (Elachouri et al., 2015 and Fridlich et al., 2009). RdCVF, but not RdCVFL, protects cone function in several genetically distinct models of RP, targeting the most debilitating step in that untreatable disease (Byrne et al., 2015, Leveillard et al., 2004 and Yang et al., 2009). Because the secondary loss of cones in retinitis pigmentosa (RP) leads to blindness, the administration of RdCVF represent a promising therapy for this untreatable retinal degenerative disease. Recently the mechanism underlying the protective role of RdCVF in RP was investigated. RdCVF acts through binding to Basigin-1 (BSG1), a transmembrane protein expressed specifically by photoreceptors. BSG1 binds to the glucose transporter GLUT1, resulting in increased glucose entry into cones (Aït-Ali et al. 2015). Identification of the remaining key actors of said mechanism would thus lead to the conception new therapy of the retinal degenerative disease.

### SUMMARY OF THE INVENTION

The present invention relates to methods and pharmaceutical compositions for the treatment of retinal degenerative diseases. In particular, the present invention is defined by the claims.

### DETAILED DESCRIPTION OF THE INVENTION

The inventors identified a new key actor of the mechanism underlying the protective role of RdCVF: 6-phosphofructo-2-kinase/fructose-2,6-bisphosphatase 2 (PFKFB2). This identification paves the way of new strategies for the treatment of retinal degenerative diseases.

Accordingly, the present invention relates to a method of treating a retinal degenerative disease in a subject in need thereof comprising administering to the subject a therapeutically effective amount of a polynucleotide encoding for the PFKFB2 kinase domain.

As used herein, the term “treatment” or “treat” refer to both prophylactic or preventive treatment as well as curative or disease modifying treatment, including treatment of patient at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

As used herein the term “retinal degenerative diseases” encompasses all retinal diseases associated with cone degeneration. The method of the present invention is thus particularly suitable for preventing cone degeneration. In particular, the retinal degenerative disease is cone dystrophy. As used herein the term “cone dystrophy” has its general meaning in the art and refers to an ocular disorder characterized by the loss of cone cells, the photoreceptors responsible for both central and color vision. The most common symptoms of cone dystrophy are vision loss (age of onset ranging from the late teens to the sixties), sensitivity to bright lights, and poor color vision. Thus the present invention is thus suitable for preventing vision loss of a patient suffering from a retinal degenerative disease. Particular examples of retinal degenerative diseases include retinitis pigmentosa (RP), Leber congenital amaurosis (LCA), age-related macular degeneration (AMD), recessive RP, dominant RP, X-linked RP, incomplete X-linked RP, dominant, dominant LCA, recessive ataxia, posterior column with RP, recessive RP with para-arteriolar preservation of the RPE, RP 12, Usher syndrome, dominant retinitis pigmentosa with sensorineural deafness, recessive retinitis punctata albescens, recessive Alstrδm syndrome, recessive Bardet-Biedl syndrome, dominant spinocerebellar ataxia w/ macular dystrophy or retinal degeneration, Recessive abetalipoproteinemia, recessive retinitis pigmentosa with macular degeneration, recessive Refsum disease adult form, recessive Refsum disease infantile form, recessive enhanced S-cone syndrome, RP with mental retardation, RP with myopathy, recessive Newfoundland rod-cone dystrophy, RetRP sinpigmento, sector RP, regional RP, Senior-Loken syndrome, Joubert syndrome, Stargardt disease juvenile, Stargardt disease late onset, dominant macular dystrophy Stargardt type, dominant Stargardt-like macular dystrophy, recessive macular dystrophy, recessive fundus flavimaculatus, recessive cone-rod dystrophy, X-linked progressive cone-rod dystrophy, dominant cone-rod dystrophy, cone-rod dystrophy; de Grouchy syndrome, dominant cone dystrophy, X-linked cone dystrophy, recessive cone dystrophy, recessive cone dystrophy with supernormal rod electroretinogram, X-linked atrophic macular dystrophy, X-linked retinoschisis, dominant macular dystrophy, dominant radial, macular drusen, dominant macular dystrophy, bull's-eye, dominant macular dystrophy butterfly-shaped, dominant adult vitelliform macular dystrophy, dominant macular dystrophy North Carolina type, dominant retinal-cone dystrophy 1, dominant macular dystrophy cystoid, dominant macular dystrophy, atypical vitelliform, foveomacular atrophy, dominant macular dystrophy Best type, dominant macular dystrophy North Carolina-like with progressive, recessive macular dystrophy juvenile with hypotrichosis, recessive foveal hypoplasia and anterior segment dysgenesis, recessive delayed cone adaptation, macular dystrophy in blue cone monochromacy, macular pattern dystrophy with type II diabetes and deafness, Flecked retina of Kandori, pattern dystrophy, dominant Stickler syndrome, dominant Marshall syndrome, dominant vitreoretinal degeneration, dominant familial exudative vitreoretinopathy, dominant vitreoretinochoroidopathy; dominant neovascular inflammatory vitreoretinopathy, Goldmann-Favre syndrome, recessive achromatopsia, dominant tritanopia, recessive rod monochromacy, congenital red-green deficiency, deuteranopia, protanopia, deuteranomaly, protanomaly, recessive Oguchi disease, dominant macular dystrophy late onset, recessive gyrate atrophy, dominant atrophia areata, dominant central areolar choroidal dystrophy, X-linked choroideremia, choroidal atrophy, central areolar, central, peripapillary, dominant progressive bifocal chorioretinal atrophy, progresive bifocal choroioretinal atrophy, dominant Doyne honeycomb retinal degeneration (Malattia Leventinese), amelogenesis imperfecta, recessive Bietti crystalline corneoretinal dystrophy, dominant hereditary vascular retinopathy with Raynaud phenomenon and migraine, dominant Wagner disease and erosive vitreoretinopathy, recessive microphthalmos and retinal disease syndrome; recessive nanophthalmos, recessive retardation, spasticity and retinal degeneration, recessive Bothnia dystrophy, recessive pseudoxanthoma elasticum, dominant pseudoxanthoma elasticum; recessive Batten disease (ceroid-lipofuscinosis), juvenile, dominant Alagille syndrome, McKusick-Kaufman syndrome, hypoprebetalipoproteinemia, acanthocytosis, palladial degeneration; Recessive Hallervorden-Spatz syndrome; dominant Sorsby's fundus dystrophy, Oregon eye disease, Kearns-Sayre syndrome, RP with developmental and neurological abnormalities, Basseb Korenzweig Syndrome, Hurler disease, Sanfilippo disease, Scieie disease, melanoma associated retinopathy, Sheen retinal dystrophy, Duchenne macular dystrophy, Becker macular dystrophy, Birdshot Retinochoroidopathy, multiple evanescent white-dot syndrome, acute zonal occult outer retinopathy, retinal vein occlusion, retinal artery occlusion, diabetic retinopathy, retinal toxicity, retinal injury, retinal traumata and retinal laser lesions, and Fundus Albipunctata, retinal detachment, diabetic retinopathy, retinopathy of prematurity.

As used herein the term “PFKFB2” has its general meaning in the art and refers to the 6-phosphofructo-2-kinase/fructose-2,6-biphosphatase 2 encoded by the PFKFB2 gene (Gene ID: 5208). PFKFB2 is involved in both the synthesis and degradation of fructose-2,6-bisphosphate, a regulatory molecule that controls glycolysis in eukaryotes and has a 6-phosphofructo-2-kinase activity that catalyzes the synthesis of fructose-2,6-bisphosphate, and a fructose-2,6-biphosphatase activity that catalyzes the degradation of fructose-2,6-bisphosphate. An exemplary amino acid sequence of PFKFB2 is reported at NCBI accession number NP 006203.2 and is represented by SEQ ID NO:1.

SEQ ID NO: 1
MSGASSSEQN NNSYETKTPN LRMSEKKCSW ASYMTNSPTL
IVMIGLPARG KTYVSKKLTR YLNWIGVPTK VFNLGVYRRE
AVKSYKSYDF FRHDNEEAMK IRKQCALVAL EDVKAYLTEE
NGQIAVFDAT NTTRERRDMI LNFAEQNSFK VFFVESVCDD
PDVIAANILE VKVSSPDYPE RNRENVMEDF LKRIECYKVT
YRPLDPDNYD KDLSFIKVIN VGQRFLVNRV QDYIQSKIVY
YLMNIHVQPR TIYLCRHGES EFNLLGKIGG DSGLSVRGKQ
FAQALRKFLE EQEITDLKVW TSQLKRTIQT AESLGVPYEQ
WKILNEIDAG VCEEMTYAEI EKRYPEEFAL RDQEKYLYRY
PGGESYQDLV QRLEPVIMEL ERQGNVLVIS HQAVMRCLLA
YFLDKGADEL PYLRCPLHTI FKLTPVAYGC KVETIKLNVE
AVNTHRDKPT NNFPKNQTPV RMRRNSFTPL SSSNTIRRPR
NYSVGSRPLK PLSPLRAQDM QEGAD

As used herein, the term “PFKFB2 kinase domain” refers to the domain responsible for the kinase activity of PFKFB2. Typically, said domain corresponds to the amino acid residue at position 1 to the amino acid residue at position 247 in SEQ ID NO:1 and is represented by SEQ ID NO: 2 (see FIG. 1).

SEQ ID NO:2
MSGASSSEQNNNSYETKTPNLRMSEKKCSWASYMTNSPTLIVMIGLPARG
KTYVSKKLTRYLNWIGVPTKVFNLGVYRREAVKSYKSYDFFRHDNEEAMK
IRKQCALVALEDVKAYLTEENGQIAVFDATNTTRERRDMILNFAEQNSFK
VFFVESVCDDPDVIAANILEVKVSSPDYPERNRENVMEDFLKRIECYKVT
YRPLDPDNYDKDLSFIKVINVGQRFLVNRVQDYIQSKIVYYLMNIHVQ

In some embodiments, the polynucleotide of the present invention encodes for an amino acid sequence having at least 80% of identity with SEQ ID NO:2.

According to the invention a first amino acid sequence having at least 80% of identity with a second amino acid sequence means that the first sequence has 80; 81; 82; 83; 84; 85; 86; 87; 88; 89; 90; 91; 92; 93; 94; 95; 96; 97; 98; 99 or 100% of identity with the second amino acid sequence. Sequence identity is frequently measured in terms of percentage identity (or similarity or homology); the higher the percentage, the more similar are the two sequences. Methods of alignment of sequences for comparison are well known in the art. Various programs and alignment algorithms are described in: Smith and Waterman, Adv. Appl. Math., 2:482, 1981; Needleman and Wunsch, J. Mol. Biol., 48:443, 1970; Pearson and Lipman, Proc. Natl. Acad. Sci. U.S.A., 85:2444, 1988; Higgins and Sharp, Gene, 73:237-244, 1988; Higgins and Sharp, CABIOS, 5:151-153, 1989; Corpet et al. Nuc. Acids Res., 16:10881-10890, 1988; Huang et al., Comp. Appls Biosci., 8:155-165, 1992; and Pearson et al., Meth. Mol. Biol., 24:307-31, 1994). Altschul et al., Nat. Genet., 6:119-129, 1994, presents a detailed consideration of sequence alignment methods and homology calculations. By way of example, the alignment tools ALIGN (Myers and Miller, CABIOS 4:11-17, 1989) or LFASTA (Pearson and Lipman, 1988) may be used to perform sequence comparisons (Internet Program® 1996, W. R. Pearson and the University of Virginia, fasta20u63 version 2.0u63, release date December 1996). ALIGN compares entire sequences against one another, while LFASTA compares regions of local similarity. These alignment tools and their respective tutorials are available on the Internet at the NCSA Website, for instance. Alternatively, for comparisons of amino acid sequences of greater than about 30 amino acids, the Blast 2 sequences function can be employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). The BLAST sequence comparison system is available, for instance, from the NCBI web site; see also Altschul et al., J. Mol. Biol., 215:403-410, 1990; Gish. & States, Nature Genet., 3:266-272, 1993; Madden et al. Meth. Enzymol., 266:131-141, 1996; Altschul et al., Nucleic Acids Res., 25:3389-3402, 1997; and Zhang & Madden, Genome Res., 7:649-656, 1997.

In some embodiments, the polynucleotide of the present invention encodes for an amino acid sequence having at least 70% of identity with SEQ ID NO:1 (i.e. a sequence having 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100% of identity with SEQ ID NO:1).

As used herein, the term “polynucleotide” has its general meaning in the art and refers to a DNA or RNA molecule. However, the term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxylmethyl) uracil, 5-fiuorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyamino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

In some embodiments, the polynucleotide of the present invention comprises the nucleic acid sequence SEQ ID NO:3 (FIG. 1) or a codon-optimized sequence thereof. As used herein, the term “codon optimized” means that a codon that expresses a bias for human (i.e. is common in human genes but uncommon in other mammalian genes or non-mammalian genes) is changed to a synonymous codon (a codon that codes for the same amino acid) that does not express a bias for human. Thus, the change in codon does not result in any amino acid change in the encoded protein. In some embodiments, the polynucleotide comprises a nucleic sequence having at least 50% of identity with SEQ ID NO:3 (i.e. a sequence having 50; 51; 52; 53; 54; 55; 56; 57; 58; 59; 60; 61; 62; 63; 64; 65; 66; 67; 68; 69; 70; 71; 72; 73; 74; 75; 76; 77; 78; 79; 80; 81; 82; 83; 84; 85; 86; 87; 88; 89; 90; 91; 92; 93; 94; 95; 96; 97; 98; 99; or 100% of identity with SEQ ID NO:3).

SEQ ID NO: 3
ATGTCTGGGGCATCTTCCTCAGAACAGAACAACAACAGCTATGAAACCAAA
ACCCCAAATCTTCGAATGTCAGAGAAGAAATGTTCATGGGCCGCCTACATG
ACCAACTCCCCGACTCTGATCGTTATGATTGGTTTGCCAGCCCGGGGTAAA
ACCTACGTGTCCAAGAAACTAACACGCTACCTCAACTGGATTGGAGTCCCC
ACCAAAGTGTTTAATCTTGGGGTGTATCGGCGTGAAGCAGTCAAGTCCTAT
AAGTCCTACGACTTCTTTCGGCATGACAATGAGGAGGCCATGAAGATCCGC
AAACAGTGTGCTCTGGTGGCGCTGGAAGATGTTAAGGCGTATCTCACTGAG
GAGAATGGTCAGATTGCGGTGTTTGATGCCACCAATACAACCCGGGAGAGG
AGGGACATGATTTTGAACTTTGCTGAACAGAATTCCTTCAAGGTATTCTTT
GTGGAATCCGTCTGTGATGATCCTGATGTCATTGCTGCCAATATTCTGGAG
GTTAAGGTATCAAGCCCTGACTATCCTGAAAGGAACAGAGAGAACGTGATG
GAGGACTTCCTGAAGAGAATTGAATGCTACAAAGTTACCTACCGACCTCTT
GACCCAGACAACTATGACAAGGATCTTTCTTTCATCAAGGTGATAAACGTG
GGCCAGCGATTTTTAGTCAACAGAGTCCAGGACTACATCCAGAGCAAGATA
GTCTACTACCTCATGAATATCCACGTCCAGTAA

In some embodiments, the polynucleotide of the present invention is included in a suitable vector.

As used herein, the term “vector” has its general meaning in the art and refers to a nucleic acid molecule into which an exogenous or heterologous or engineered nucleic acid transgene may be inserted. Vectors preferably have one or more origin of replication, and one or more site into which the recombinant DNA can be inserted. Common vectors include plasmids, viral genomes, and (primarily in yeast and bacteria) “artificial chromosomes.” “Virus vectors” are defined as replication defective viruses containing the exogenous or heterologous nucleic acid transgene(s). More particularly, the vector is a viral vector. As used herein, the term “viral vector” refers to a synthetic or recombinant viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In some embodiments, the genome of the viral vector does not include genes encoding the enzymes required to replicate (the genome can be engineered to be “gutless”—containing only the transgene of interest flanked by the signals required for amplification and packaging of the artificial genome), but these genes may be supplied during production.

In some embodiments, the viral vector is an AAV. As used herein the term “AAV” refers to the more than 30 naturally occurring and available adeno-associated viruses, as well as artificial AAVs. Typically the AAV capsid, ITRs, and other selected AAV components described herein, may be readily selected from among any AAV, including, without limitation, AAV1, AAV2, AAV3, AAV4, AAVS, AAV6, AAV6.2, AAV7, AAV8, AAV9, rh10, AAVrh64R1, AAVrh64R2, rh8, rh.10, variants of any of the known or mentioned AAVs or AAVs yet to be discovered or variants or mixtures thereof. See, e.g., WO 2005/033321. The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, Va.). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc. As used herein, “artificial AAV” means, without limitation, an AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In some embodiments, the AAV is AAV2/5 and AAV2/8. Methods for generating and isolating AAV viral vectors suitable for delivery to a subject are known in the art. See, e.g., U.S. Pat. Nos. 7,790,449; 7,282,199; WO 2003/042397; WO 2005/033321, WO 2006/110689; and U.S. Pat. No. 7,588,772 B2]. In a one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. More recently, systems have been developed that do not require infection with helper virus to recover the AAV—the required helper functions (i.e., adenovirus E1, E2a, VA, and E4 or herpesvirus ULS, UL8, UL52, and UL29, and herpesvirus polymerase) are also supplied, in trans, by the system. In these newer systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al., 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065. See generally, e.g., Grieger & Samulski, 2005, “Adeno-associated virus as a gene therapy vector: Vector development, production and clinical applications,” Adv. Biochem. Engin/Biotechnol. 99: 119-145; Buning et al., 2008, “Recent developments in adeno-associated virus vector technology,” J. Gene Med. 10:717-733; and the references cited below, each of which is incorporated herein by reference in its entirety.

In some embodiments, the vector of the present invention include a promoter sequence as part of the expression control sequences. In some embodiments, a suitable promoter is a hybrid chicken β-actin (CBA) promoter with cytomegalovirus (CMV) enhancer elements. Still other suitable promoters are the CB7 promoter, as well such as viral promoters, constitutive promoters, regulatable promoters [see, e.g., WO 2011/126808 and WO 2013/04943], or a promoter responsive to physiologic cues may be used may be utilized in the in the vector. In some embodiments, the promoter is cell-specific. The term “cell-specific” means that the particular promoter selected for the recombinant vector can direct expression of the polynucleotide of the present invention in a particular cell. In some embodiments, the promoter is specific for expression of the polynucleotide in photoreceptor cells. In some embodiments, the promoter is specific for expression in cones. Exemplary promoters may be the human G-protein-coupled receptor protein kinase 1 (GRK1) promoter (Genbank Accession number AY327580). In some embodiments, the promoter is a 292 nt fragment (positions 1793-2087) of the GRK1 promoter (See, Beltran et al, Gene Therapy 2010 17:1162-74, which is hereby incorporated by reference herein). In some embodiments, the promoter is the human interphotoreceptor retinoid-binding protein proximal (IRBP) promoter. In some embodiments, the promoter is a 235 nt fragment of the hIRBP promoter. In some embodiments, the promoter is the RPGR proximal promoter (Shu et al, IOVS, May 2102, which is incorporated by reference herein). Other promoters useful in the invention include, without limitation, the rod opsin promoter, the red-green opsin promoter, the blue opsin promoter, the cGMP-β-phosphodiesterase promoter, the mouse opsin promoter (Beltran et al 2010 cited above), the rhodopsin promoter (Mussolino et al, Gene Ther, July 2011, 18(7):637-45); the alpha-subunit of cone transducin (Morrissey et al, BMC Dev, Biol, January 2011, 11:3); beta phosphodiesterase (PDE) promoter; the retinitis pigmentosa (RP1) promoter (Nicord et al, J. Gene Med, December 2007, 9(12):1015-23); the NXNL2/NXNL1 promoter (Lambard et al, PLoS One, October 2010, 5(10):e13025), the retinal degeneration slow/peripherin 2 (Rds/perph2) promoter (Cai et al, Exp Eye Res. 2010 August; 91(2):186-94); and the VMD2 promoter (Kachi et al, Human Gene Therapy, 2009 (20:31-9)).

As used herein the term “administering” means delivering the polynucleotide to the target selected cells, in particular cones. Typical routes of administration typically include systemic routes, e.g., intraarterial, intraocular, intravenous, intramuscular, subcutaneous, intradermal, and other parental routes of administration. Direct delivery to the eye optionally via ocular delivery, intra-retinal injection, intravitreal, topical represent a particular interest for the treatment of the retinal degenerative diseases. Routes of administration may be combined, if desired. In some embodiments, the administration is repeated periodically. The polynucleotide of the present invention may be delivered in a single composition or multiple compositions. Optionally, two or more different AAV may be delivered, or multiple viruses [see, e.g., WO20 2011/126808 and WO 2013/049493]. In some embodiments, multiple viruses may contain different replication-defective viruses (e.g., AAV and adenovirus), alone or in combination with proteins.

By a “therapeutically effective amount” is meant a sufficient amount of the polynucleotide of the present invention for the treatment of the retinal degenerative disease at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, preferably from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

According to the invention, the polynucleotide of the present invention typically inserted in a viral vector is administered to the subject in the form of a pharmaceutical composition. Typically, the polynucleotide of the present invention may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. In the pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polynucleotide of the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin. Sterile injectable solutions are prepared by incorporating the active compounds in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the typical methods of preparation are vacuum-drying and freeze-drying techniques which yield a powder of the active ingredient plus any additional desired ingredient from a previously sterile-filtered solution thereof. The preparation of more, or highly concentrated solutions for direct injection is also contemplated, where the use of DMSO as solvent is envisioned to result in extremely rapid penetration, delivering high concentrations of the active agents to a small tumor area. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. Some variation in dosage will necessarily occur depending on the condition of the subject being treated. The person responsible for administration will, in any event, determine the appropriate dose for the individual subject.

The invention will be further illustrated by the following figures and examples. However, these examples and figures should not be interpreted in any way as limiting the scope of the present invention.

### FIGURES

FIG. 1: sequence of PFKFB2 kinase domain.

FIG. 2: PFKFB2 is expressed by cones in a rod-dependant manner. A. Expression of PFKFB2 in the heart, brain and retina on the wild-type mouse by western blotting. B. Differential expression of PFKFB2 in the wild-type and rd1 retina at post natal day 21 and 35 by western blotting. C. quantification of the signal. D. Expression of Rho in the wild-type and rd1 by microarray analysis. By postnatal day 21, the majority of rods have degenerated.

FIG. 3:A. The expression of Pfkfb2 is induced by glucose in the cone-enriched cultures from chicken embryo. B. Electroporation of mouse Pfkfb2 cDNA plasmid increases cone survival in the cone-enriched cultures from chicken embryo.

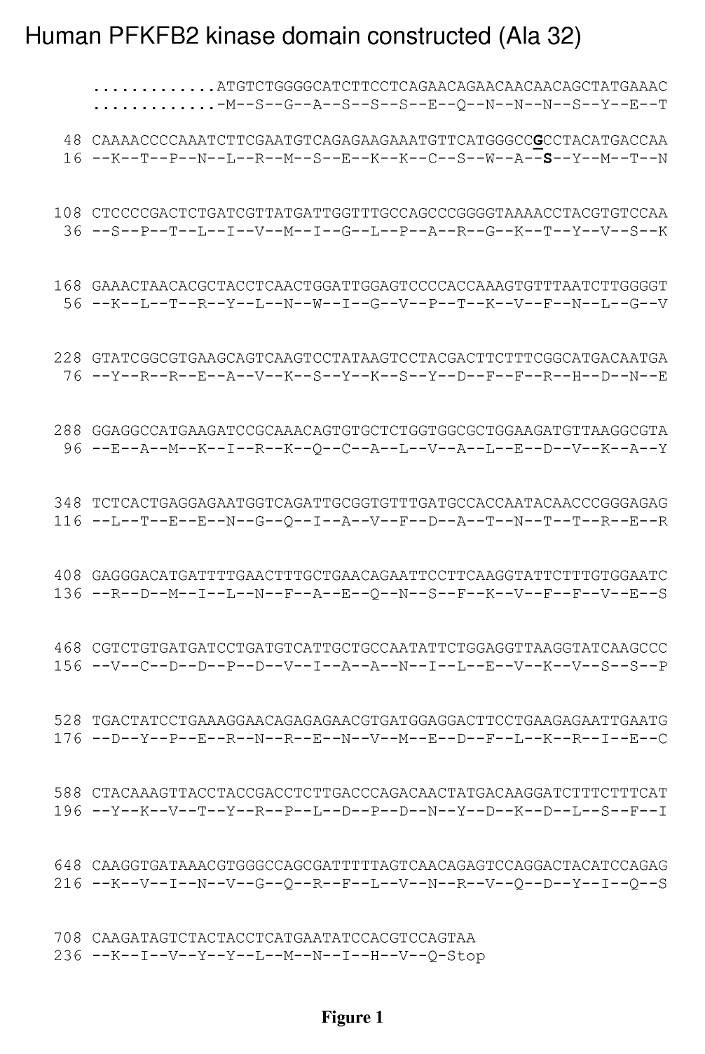

### EXAMPLE

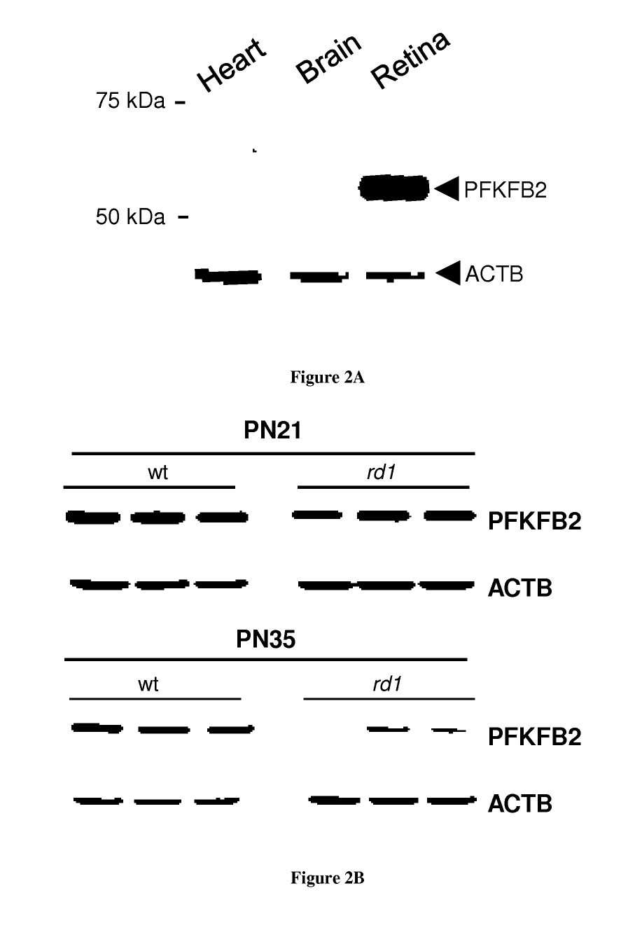

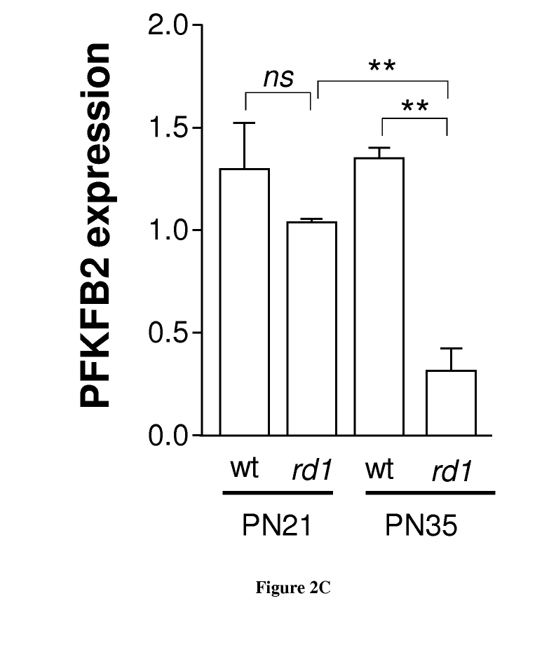

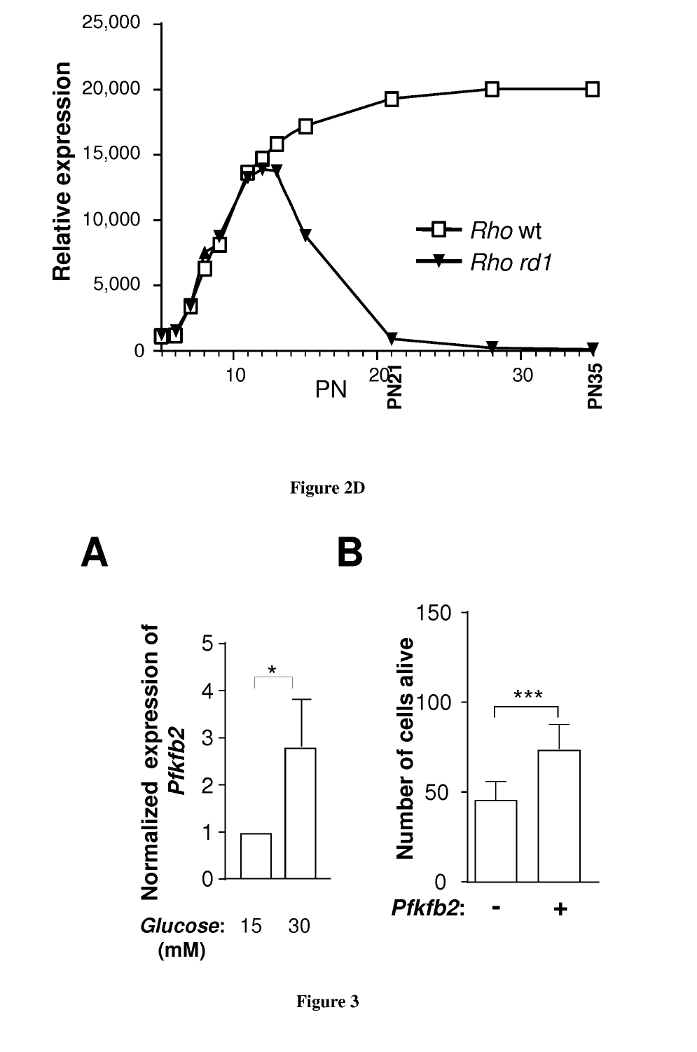

In the prior art, PFKFB2 is described as being expressed mainly in the heart. However we showed unexpectedly that this protein is expressed in high amount in retina in comparison with heart (FIG. 2A). We further showed that PFKFB2 is expressed by cones in a rod-dependant manner. In particular, we showed that its expression follows the viability of cones (FIGS. 2B-D): its expression is lost a day 35 in an animal model retinitis pigmentosa (rd1 mouse). The expression of PFKFB2 is induced by glucose (FIG. 3A) and we accumulate evidences that this protein, especially its kinase domain, is involved in the mechanism of action of RdCVF. More particularly we showed that transduction of a polynucleotide encoding for PFKFB2 increases cone survival (FIG. 3A). We now perform experiments in the rd1 animal model with AAV (AAV2.8) vectors comprising the polynucleotide encoding for the PFKFB2 kinase domain.

### REFERENCES

Throughout this application, various references describe the state of the art to which this invention pertains. The disclosures of these references are hereby incorporated by reference into the present disclosure.
Aït-Ali N, Fridlich R, Millet-Puel G, Clérin E, Delalande F, Jaillard C, Blond F, Perrocheau L, Reichman S, Byrne L C, Olivier-Bandini A, Bellalou J, Moyse E, Bouillaud F, Nicol X, Dalkara D, van Dorsselaer A, Sahel J A, Leveillard T. Rod-derived cone viability factor promotes cone survival by stimulating aerobic glycolysis. Cell. 2015 May 7; 161(4):817-32.
T. Léveillard, S. Mohand-Saïd, O. Lorentz, D. Hicks, A. C. Fintz, E. Clérin, M. Simonutti, V. Forster, N. Cavusoglu, F. Chalmel, et al. Identification and characterization of rod-derived cone viability factor Nat. Genet., 36 (2004), pp. 755-759
G. Elachouri, I. Lee-Rivera, E. Clerin, M. Argentini, R. Fridlich, F. Blond, V. Ferracane, Y. Yang, W. Raffelsberger, J. Wan, et al. The thioredoxin RdCVFL protects against photo-oxidative retinal damage Free Radic. Biol. Med., 81 (2015), pp. 22-29.
R. Fridlich, F. Delalande, C. Jaillard, J. Lu, L. Poidevin, T. Cronin, L. Perrocheau, G. Millet-Puel, M. L. Niepon, O. Poch, et al. The thioredoxin-like protein rod-derived cone viability factor (RdCVFL) interacts with TAU and inhibits its phosphorylation in the retina Mol. Cell. Proteomics, 8 (2009), pp. 1206-1218.
L. C. Byrne, D. Dalkara, G. Luna, S. K. Fisher, E. Clérin, J. A. Sahel, T. Léveillard, J. G. Flannery Viral-mediated RdCVF and RdCVFL expression protects cone and rod photoreceptors in retinal degeneration J. Clin. Invest., 125 (2015), pp. 105-116
Y. Yang, S. Mohand-Said, A. Danan, M. Simonutti, V. Fontaine, E. Clerin, S. Picaud, T. Leveillard, J. A. Sahel Functional cone rescue by RdCVF protein in a dominant model of retinitis pigmentosa Mol. Ther., 17 (2009), pp. 787-795.

1. A method of treating a retinal degenerative disease in a subject in need thereof comprising administering to the subject a therapeutically effective amount of a polynucleotide encoding for the PFKFB2 kinase domain.

2. The method of claim 1 wherein the retinal degenerative disease is a cone dystrophy.

3. The method of claim 1 wherein the retinal degenerative disease is selected from the group consisting of retinitis pigmentosa (RP), Leber congenital amaurosis (LCA), age-related macular degeneration (AMD), recessive RP, dominant RP, X-linked RP, incomplete X-linked RP, dominant, dominant LCA, recessive ataxia, posterior column with RP, recessive RP with para-arteriolar preservation of the RPE, RP 12, Usher syndrome, dominant retinitis pigmentosa with sensorineural deafness, recessive retinitis punctata albescens, recessive Alstrδm syndrome, recessive Bardet-Biedl syndrome, dominant spinocerebellar ataxia w/ macular dystrophy or retinal degeneration, Recessive abetalipoproteinemia, recessive retinitis pigmentosa with macular degeneration, recessive Refsum disease adult form, recessive Refsum disease infantile form, recessive enhanced S-cone syndrome, RP with mental retardation, RP with myopathy, recessive Newfoundland rod-cone dystrophy, RetRP sinpigmento, sector RP, regional RP, Senior-Loken syndrome, Joubert syndrome, Stargardt disease juvenile, Stargardt disease late onset, dominant macular dystrophy Stargardt type, dominant Stargardt-like macular dystrophy, recessive macular dystrophy, recessive fundus flavimaculatus, recessive cone-rod dystrophy, X-linked progressive cone-rod dystrophy, dominant cone-rod dystrophy, cone-rod dystrophy; de Grouchy syndrome, dominant cone dystrophy, X-linked cone dystrophy, recessive cone dystrophy, recessive cone dystrophy with supernormal rod electroretinogram, X-linked atrophic macular dystrophy, X-linked retinoschisis, dominant macular dystrophy, dominant radial, macular drusen, dominant macular dystrophy, bull's-eye, dominant macular dystrophy butterfly-shaped, dominant adult vitelliform macular dystrophy, dominant macular dystrophy North Carolina type, dominant retinal-cone dystrophy 1, dominant macular dystrophy cystoid, dominant macular dystrophy, atypical vitelliform, foveomacular atrophy, dominant macular dystrophy Best type, dominant macular dystrophy North Carolina-like with progressive, recessive macular dystrophy juvenile with hypotrichosis, recessive foveal hypoplasia and anterior segment dysgenesis, recessive delayed cone adaptation, macular dystrophy in blue cone monochromacy, macular pattern dystrophy with type II diabetes and deafness, Flecked retina of Kandori, pattern dystrophy, dominant Stickler syndrome, dominant Marshall syndrome, dominant vitreoretinal degeneration, dominant familial exudative vitreoretinopathy, dominant vitreoretinochoroidopathy; dominant neovascular inflammatory vitreoretinopathy, Goldmann-Favre syndrome, recessive achromatopsia, dominant tritanopia, recessive rod monochromacy, congenital red-green deficiency, deuteranopia, protanopia, deuteranomaly, protanomaly, recessive Oguchi disease, dominant macular dystrophy late onset, recessive gyrate atrophy, dominant atrophia areata, dominant central areolar choroidal dystrophy, X-linked choroideremia, choroidal atrophy, central areolar, central, peripapillary, dominant progressive bifocal chorioretinal atrophy, progresive bifocal choroioretinal atrophy, dominant Doyne honeycomb retinal degeneration (Malattia Leventinese), amelogenesis imperfecta, recessive Bietti crystalline corneoretinal dystrophy, dominant hereditary vascular retinopathy with Raynaud phenomenon and migraine, dominant Wagner disease and erosive vitreoretinopathy, recessive microphthalmos and retinal disease syndrome; recessive nanophthalmos, recessive retardation, spasticity and retinal degeneration, recessive Bothnia dystrophy, recessive pseudoxanthoma elasticum, dominant pseudoxanthoma elasticum; recessive Batten disease (ceroid-lipofuscinosis), juvenile, dominant Alagille syndrome, McKusick-Kaufman syndrome, hypoprebetalipoproteinemia, acanthocytosis, palladial degeneration; Recessive Hallervorden-Spatz syndrome; dominant Sorsby's fundus dystrophy, Oregon eye disease, Kearns-Sayre syndrome, RP with developmental and neurological abnormalities, Basseb Korenzweig Syndrome, Hurler disease, Sanfilippo disease, Scieie disease, melanoma associated retinopathy, Sheen retinal dystrophy, Duchenne macular dystrophy, Becker macular dystrophy, Birdshot Retinochoroidopathy, multiple evanescent white-dot syndrome, acute zonal occult outer retinopathy, retinal vein occlusion, retinal artery occlusion, diabetic retinopathy, retinal toxicity, retinal injury, retinal traumata and retinal laser lesions, and Fundus Albipunctata, retinal detachment, diabetic retinopathy, and retinopathy of prematurity.

4. The method of claim 1 wherein the retinal degenerative disease is retinitis pigmentosa.

5. The method of claim 1 wherein the polynucleotide encodes for an amino acid sequence having at least 80% of identity with SEQ ID NO:2.

6. The method of claim 1 wherein the polynucleotide encodes for an amino acid sequence having at least 70% of identity with SEQ ID NO:1.

7. The method of claim 1 wherein the polynucleotide comprises the nucleic acid sequence SEQ ID NO:3 or a codon-optimized sequence thereof.

8. The method of claim 1 wherein the polynucleotide comprises a nucleic sequence having at least 50% of identity with SEQ ID NO:3.

9. The method of claim 1 wherein the polynucleotide is included in a suitable vector.

10. The method of claim 9 wherein the vector is a viral vector such as an AVV.

11. The method of claim 10 wherein the AAV is AAV2/5 and AAV2/8.

12. The method of claim 9 wherein the vector includes a promoter sequence as part of the expression control sequences.

13. The method of claim 12 wherein the promoter is specific for expression in cones.

14. The method of claim 13 wherein the polynucleotide is delivered to the eye optionally via ocular, intra-retinal injection, intravitreal, or topical delivery.


SEQUENCE LISTING (3 sequences)

SEQ ID NO: 1 - protein, 505 aa, Homo sapiens
MSGASSSEQNNNSYETKTPNLRMSEKKCSWASYMTNSPTLIVMIGLPARGKTYVSKKLTRYLNWIGVPTKVFNLGVYRREAVKSYKSYDFFRHDNEEAMKIRKQCALVALEDVKAYLTEENGQIAVFDATNTTRERRDMILNFAEQNSFKVFFVESVCDDPDVIAANILEVKVSSPDYPERNRENVMEDFLKRIECYKVTYRPLDPDNYDKDLSFIKVINVGQRFLVNRVQDYIQSKIVYYLMNIHVQPRTIYLCRHGESEFNLLGKIGGDSGLSVRGKQFAQALRKFLEEQEITDLKVWTSQLKRTIQTAESLGVPYEQWKILNEIDAGVCEEMTYAEIEKRYPEEFALRDQEKYLYRYPGGESYQDLVQRLEPVIMELERQGNVLVISHQAVMRCLLAYFLDKGADELPYLRCPLHTIFKLTPVAYGCKVETIKLNVEAVNTHRDKPTNNFPKNQTPVRMRRNSFTPLSSSNTIRRPRNYSVGSRPLKPLSPLRAQDMQEGAD

SEQ ID NO: 2 - protein, 248 aa, Homo sapiens
MSGASSSEQNNNSYETKTPNLRMSEKKCSWASYMTNSPTLIVMIGLPARGKTYVSKKLTRYLNWIGVPTKVFNLGVYRREAVKSYKSYDFFRHDNEEAMKIRKQCALVALEDVKAYLTEENGQIAVFDATNTTRERRDMILNFAEQNSFKVFFVESVCDDPDVIAANILEVKVSSPDYPERNRENVMEDFLKRIECYKVTYRPLDPDNYDKDLSFIKVINVGQRFLVNRVQDYIQSKIVYYLMNIHVQ

SEQ ID NO: 3 - DNA, 747 bp, Homo sapiens
atgtctggggcatcttcctcagaacagaacaacaacagctatgaaaccaaaaccccaaatcttcgaatgtcagagaagaaatgttcatgggccgcctacatgaccaactccccgactctgatcgttatgattggtttgccagcccggggtaaaacctacgtgtccaagaaactaacacgctacctcaactggattggagtccccaccaaagtgtttaatcttggggtgtatcggcgtgaagcagtcaagtcctataagtcctacgacttctttcggcatgacaatgaggaggccatgaagatccgcaaacagtgtgctctggtggcgctggaagatgttaaggcgtatctcactgaggagaatggtcagattgcggtgtttgatgccaccaatacaacccgggagaggagggacatgattttgaactttgctgaacagaattccttcaaggtattctttgtggaatccgtctgtgatgatcctgatgtcattgctgccaatattctggaggttaaggtatcaagccctgactatcctgaaaggaacagagagaacgtgatggaggacttcctgaagagaattgaatgctacaaagttacctaccgacctcttgacccagacaactatgacaaggatctttctttcatcaaggtgataaacgtgggccagcgatttttagtcaacagagtccaggactacatccagagcaagatagtctactacctcatgaatatccacgtccagtaa